(19) United States
(12) Patent Application Publication
(10) Pub. No.: US 2025/0305902 A1
(43) Pub. Date: Oct. 2, 2025

(54) SYSTEMS AND METHODS FOR METHANE MONITORING

(71) Applicant: Shepherd Safety Systems, LLC, Midland, TX (US)

(72) Inventor: Seth Cameron LONG, Midland, TX (US)

(21) Appl. No.: 18/617,435

(22) Filed: Mar. 26, 2024

(51) Int. Cl. G01M 3/04; H04W 4/38

(52) U.S. Cl. CPC G01M 3/04; H04W 4/38

(57) ABSTRACT

A methane monitoring system comprising one or more methane sensors, a control hub, and a server. The one or more methane sensors are coupled to one or more pieces of equipment, the one or more methane sensors comprising an internal non-dispersive infrared sensor configured to measure an amount of methane. The control hub is in communication with the one or more sensors and the control hub is configured to receive a sensor signal corresponding to the measured amount of methane from the one or more methane sensors. The server is in communication with the control hub and the server is configured to receive information regarding the measured amount of methane from the control hub.

### BACKGROUND

### Field

Embodiments of the disclosure relate to systems and methods for methane monitoring.

### Description of the Related Art

Worksites utilize many different types of equipment in large numbers. One major concern at worksites is the possibility of a methane leak from any number of the pieces of equipment. Although there are many different types of systems and methods for monitoring methane, there is a continuous need for new and improved methane monitoring systems and methods.

### SUMMARY

A methane monitoring system comprising one or more methane sensors, a control hub, and a server. The one or more methane sensors are coupled to one or more pieces of equipment, the one or more methane sensors comprising an internal non-dispersive infrared sensor configured to measure an amount of methane. The control hub is in communication with the one or more sensors and the control hub is configured to receive sensor data corresponding to the measured amount of methane from the one or more methane sensors. The server is in communication with the control hub and the server is configured to receive information regarding the measured amount of methane from the control hub.

A method for monitoring methane, comprising: receiving one or more sensor data from one or more methane sensors comprising an internal nondispersive infrared sensor, the one or more methane sensors coupled to one or more pieces of equipment, wherein the data correspond to methane measurements taken by the one or more methane sensors; determining an amount of methane measured and a location of the one or more methane sensors measuring the amount of methane based on the one or more sensor data from the one or more methane sensors; and communicating information regarding the amount of methane and the location of the one or more methane sensors to a server.

A method for monitoring methane at a worksite comprising: coupling one or more methane sensors to one or more pieces of equipment, the one or more methane sensors comprising an internal non-dispersive infrared sensor configured to measure methane; measuring an amount of methane with the one or more methane sensors; communicating the measured amount of methane to a control hub; and communicating information regarding the measured amount of methane and a methane leak to a server.

### BRIEF DESCRIPTION OF THE DRAWINGS

So that the manner in which the above-recited features of the disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 illustrates a methane monitoring system, according to one embodiment.

FIG. 2 illustrates the one or more methane sensors, the control hub, the indicator, the one or more personal devices, and the server of the methane monitoring system of FIG. 1.

FIG. 3 illustrates the methane monitoring system of FIG. 1, according to one embodiment.

FIG. 4 illustrates a method for monitoring methane, according to one embodiment.

FIG. 5 illustrates a method for monitoring methane, according to one embodiment.

### DETAILED DESCRIPTION

The disclosure contemplates that terms such as “couples,” “coupling,” “couple,” and “coupled” may include but are not limited to welding, interference fitting, magnetic coupling, and/or fastening such as by using bolts, threaded connections, pins, clips, and/or screws. The disclosure contemplates that terms such as “couples,” “coupling,” “couple,” and “coupled” may include but are not limited to integrally forming. The disclosure contemplates that terms such as “couples,” “coupling,” “couple,” and “coupled” may include but are not limited to direct coupling and/or indirect coupling, such as indirect coupling through components such as links.

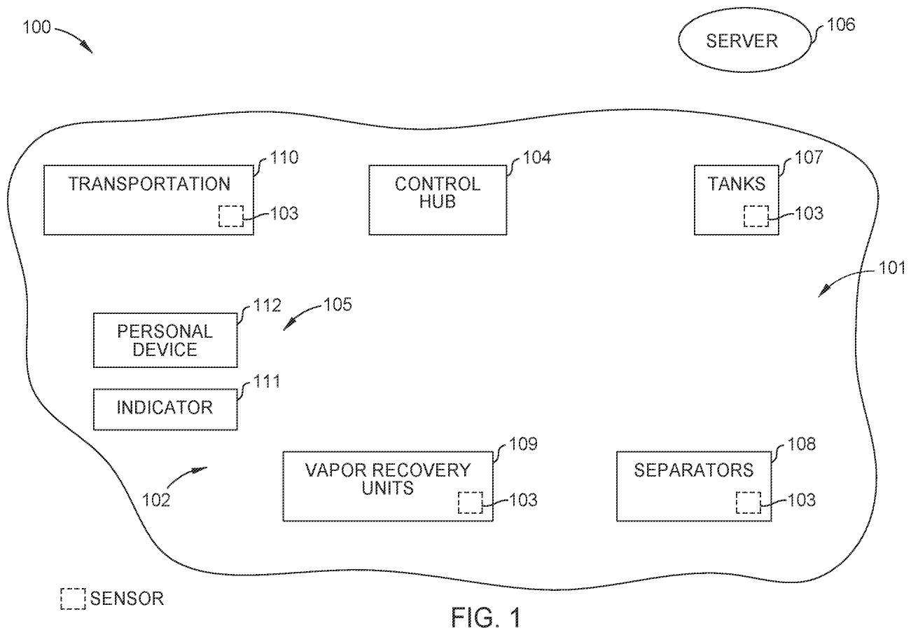

FIG. 1 illustrates a methane monitoring system 100 located at a worksite 101, according to one embodiment. The worksite 101 may be an oilfield. The worksite 101 may have facilities for the drilling, production, and/or completion of oil and/or gas wells. The worksite 101 may include facilities for transportation and/or storage of oil and/or gas. One or more pieces of equipment 102 are located at the worksite 101. The methane monitoring system 100 comprises one or more methane sensors 103 coupled to the one or more pieces of equipment 102, a control hub 104, one or more personal devices 112, an indicator 111, and a server 106.

The one or more pieces of equipment 102 may be associated with tasks of the worksite 101 such as the production of oil and/or gas. The equipment 102 may include, but is not limited to, tanks 107, separators 108, vapor recovery units 109, transportation equipment 110, and/or any combination thereof. Each of these pieces of equipment 102 may have areas of concern for methane leaks. The result of having many different types of equipment 102 at the worksite 101 with areas of concern for methane leaks is the risk of methane leaks, which may be hard to detect, locate, and/or measure.

There may be multiple of each of the tanks 107, separators 108, vapor recovery units 109, and transportation equipment 110 at the worksite 101. Each of these pieces of equipment 102 accomplishes particular tasks. The tanks 107 store fluid at the worksite 101 and may be for, but are not limited to, drilling, production, general storage purposes, and/or any combination thereof. The separators 108 are vessels used to separate oil, gas, and water from a total fluid stream produced by a well. The vapor recover units 109 consist of a system for recovering vapors formed inside completely sealed oil or condensate tanks. The transportation equipment 110 generally allows for the transportation of oil, gas, water, personnel, and/or equipment.

Each of the one or more pieces of equipment 102 may develop leaks where methane gas may leak from. A leak is an area that allows methane gas to escape from the one or more pieces of equipment 102 into the environment, such as the atmosphere. The leaks can develop due to, but not limited to, age, weather conditions, wear, improper assembly, material deterioration and/or any combination thereof. Exemplary leaks include, but are not limited to, deterioration and wear of thief hatch seals on the equipment 102, tank relief valve venting due to pressure, deterioration in materials of the vapor recovery units 109, improper assembly and wear of the equipment 102.

The one or more methane sensors 103 are located at the worksite 101 and/or on individual pieces of equipment 102, and are configured to measure methane gas surrounding the one or more methane sensors 103. The number of methane sensors 103 may be based on need for the worksite 101 and/or the equipment 102. This need can be based on likelihood of failure of the equipment 102 that would result in a leak due to conditions including, but not limited to those listed above.

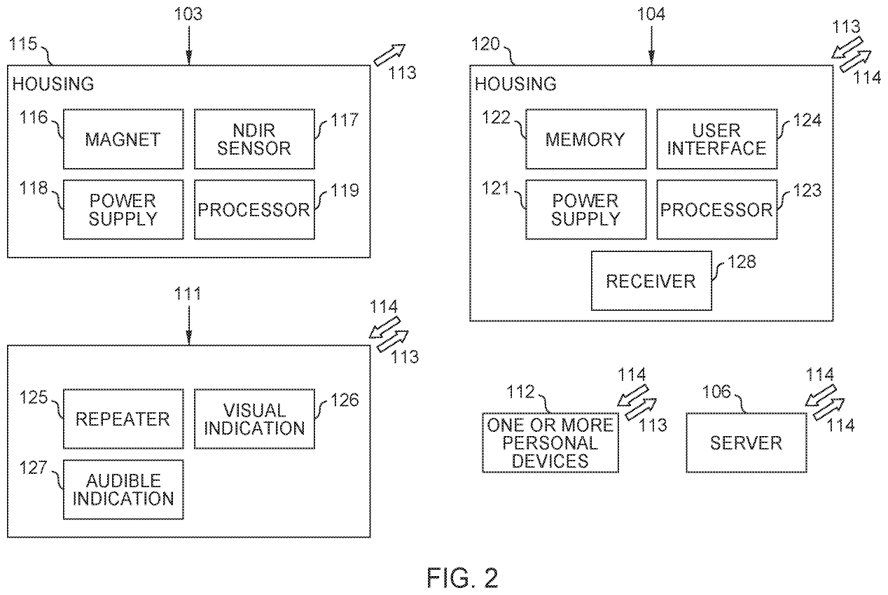

FIG. 2 illustrates the one or more methane sensors 103, the control hub 104, the indicator 111, the one or more personal devices 112, and the server 106 of the methane monitoring system 100 of FIG. 1.

The one or more methane sensors 103 include a housing 115, a magnet 116, an internal nondispersive infrared (NDIR) sensor 117, a power supply 118, and a processor 119. The housing 115 contains the other components of the one or more methane sensors 103 and is made of materials including, but not limited to, fiberglass. The magnet 116 is used to couple the one or more methane sensors 103 to and dispose the one or more methane sensors 103 on the one or more pieces of equipment 102. As discussed above, the equipment 102 at the worksite 101, such as the tanks 107, the separators 108, the vapor recovery units 109, and the transportation equipment 110 may develop methane leaks at various areas on the equipment 102. Areas of concern for these leaks may exist in locations including, but not limited to, connections to the equipment 102, weld seams, bolted seams, high stress areas, cracked areas, corroded areas, areas showing signs of degradation or wear, and/or any combination thereof.

As such, the one or more methane sensors 103 are magnetically coupled to and disposed on the equipment 102 near, on, or close to these areas of concern. In one or more embodiments, there may be more than one methane sensor 103 coupled to one or more pieces of equipment 102. In one or more embodiments, there may be only one methane sensor 103 coupled to a single piece of equipment 102 with only one area of concern. In one or more embodiments, a single piece of equipment 102 may have more than one area of concern and more than one methane sensor 103. In one or more embodiments, a single piece of equipment 102 may have more than one area of concern and only one methane sensor 103. It is desired that the one or more methane sensors 103 are coupled to the equipment 102 as close as possible to areas as concern, in some instances this may be on the area of concern or within one foot of the area of concern.

In one or more embodiments, the site 101 is evaluated prior to deployment based on size, type, terrain, and predominate wind direction. Afterwards, the equipment 102 is evaluated to determine most likely leak points (MLLP) such as those described above. After these evaluations, the site is mapped and plotted according to the MLLPs. After mapping, it is desirable to place the sensors 103 within about 1 foot of an MLLP. In one or more embodiments, the sensors 103 may be placed within about 5 feet of an MLLP. In one or more embodiments, the sensors 103 may have an operating temperature range of −20° C. to 50° C. and have an operating relative humidity range of 0%-90% continuous and 95% intermittent.

The internal NDIR sensor 117 is contained in the housing 115 and is configured to measure methane gas. Measuring methane gas includes detecting any presence of methane gas and/or measuring an amount of methane gas in volume measurements of methane gas in the environment surrounding the one or more methane sensors 103. The measurements may be in standard cubic feet per hour (SCFH) and/or may be ratio measurements of methane gas to air in the environment around the one or more methane sensors 103 in parts per million (ppm). Based on the volume and/or ratio measured, a user and/or the methane monitoring system 100 can determine if a methane gas leak is present. The NDIR sensor 117 operates similarly to the NDIR disclosed in U.S. Pat. No. 10,386,298 which is incorporated by reference herein in its entirety. In one or more embodiments, the NDIR sensor 117 may sense methane with a 0-100% lower explosive limit (LEL) percent rage, and within a range of 100 ppm-1,000,000 ppm with a resolution of 100 ppm and an accuracy of +/−100 ppm. The NDIR sensor 117 may also sense carbon dioxide (CO2) within a range of 1 ppm-1,000,000 ppm and a resolution of 500 ppm and an accuracy of +/−500 ppm.

In one or more embodiments, the NDIR sensor 117 is a smart integrated system and includes mirror optical system, photodiodes and LEDs, signal amplifiers, microcontrollers, current drivers of infrared LEDs, universal asynchronous receiver-transmitter (UART) interface signal generators and supply forming voltage units. The NDIR sensor 117 microcontrollers may perform storage of unique sensor calibration constants, processing of measurement results and concentration of measured gas, and information exchange. The NDIR sensor 117 operating principle is based on NDIR technology, e.g. on selective infrared radiation absorption by gas molecules. Infrared radiation from LED permeates through a measuring diffusion-type gas cell and arrives on signal and reference photodetectors, one of which detects radiation only in a wavelength range of infrared radiation absorbed by gases, while the other one detects radiation only in a wavelength range of 3.5 μm-3.7 μm for hydrocarbons. Gas flowing through the gas cell absorbs the radiation of the operating wavelength (As) and does not affect the radiation of the reference wavelength (λref). Amplitude of the photodetector operating and reference signals varies with the target gas concentration.

NDIR sensors generally use an infrared lamp to direct waves of light through a tube filled with a sample of air. This air moves toward an optical filter in front of an infrared light detector. The infrared light detector measures the amount of infrared light that passes through the optical filter to determine the presence of gas in the air. As the infrared light passes through the sample of air, the gas present absorbs specific bands of infrared light while letting other wavelengths of light pass through. At the detector end, the remaining light hits an optical filter that absorbs every wavelength of light except the wavelength absorbed. Finally, the infrared detector reads the remaining amount of light that was not absorbed by the gas or the optical filter. The remaining amount of light that was not absorbed by the gas or the optical filter is indicative of what gas and the amount of gas present in the sample.

The power supply 118 is configured to supply power to the NDIR sensor 117 and the processor 119. The processor 119 utilizes the NDIR sensor 117 to detect the presence of and measure an amount of methane gas. The power supply 118 may be a battery (e.g. a Lithium-Ion rechargeable battery).

The processor 119 is configured to wirelessly communicate with the control hub 104, an indicator 111, and one or more personal devices 112 via sensor signals 113 such as by an antenna of the sensor 103. The sensor signals 113 include, but is not limited to, a presence, or lack thereof, of methane gas in the environment around the one or more methane sensor 103, the measured amount of methane gas in the environment around the one or more methane sensor 103, an identifier indicating which of the one or more methane sensors 103 is sending the sensor signals 113, and/or any combination thereof.

The one or more methane sensors 103 may be certified for Class 1, Division 1 (Class 1, Div. 1) locations by global service providers Eurofins MET Labs and/or UL (Underwriters Laboratories). Class 1, Div. 1 locations are locations 1) in which ignitable concentrations of flammable gases or vapors exist continuously, intermittently, or periodically under normal operating conditions; or 2) in which ignitable concentration of such gases or vapors may exist frequently because of repair or maintenance operations or because of leakage; or 3) in which breakdown or faulty operation of equipment or processes might release ignitable concentrations of flammable gases or vapors, and might also cause simultaneous failure of electrical equipment. For example, Class 1 Div. 1 certification tests on the methane sensors 103 were conducted in accordance with UL 61010-1/CSA C22.2 No. 61010-1, Third Edition: Safety requirements for electrical equipment for measurement, control, and laboratory use Part 1: General requirements, Dated May 11, 2012. For example, Class 1 Div. 1 certification tests on the methane sensors 103 were conducted in accordance with UL 913 Intrinsically Safe Apparatus and Associated Apparatus for use in Class I, II, and III, Division 1, Hazardous (Classified) Locations. Eighth Edition, Dated Dec. 6, 2013.

The control hub 104 acts as a controller and is located at the worksite 101 or near the worksite 101 such that the control hub 104 is within communication range of the one or more methane sensors 103, the indicator 111, and the one or more personal devices 112.

The control hub 104 comprises a housing 120, a power supply 121, a memory 122, a processor 123, a user interface 124, and a receiver 128. The housing 120 may be made of fiberglass, and contains the other components of the control hub 104. The receiver 128 may be an antenna. The user interface 124 may comprise a touch screen that may display messages or allow interactivity with a user to configure settings. The power supply 121 is configured to power the other components of the control hub 104. The receiver 128 is configured to receive sensor signals 113 from the one or more methane sensors 103. The processor 122 is configured to analyze the sensor signals 113 and wirelessly communicate with the indicator 111, the one or more personal devices 112, and the server 106.

The receiver 128 receives the sensor signals 113. The processor 123 then decodes the sensor signals 113, analyzes the data from the decoded sensor signals 113, determines a location of the one or more methane sensors 103 sending the sensor signals 113, stores the raw and/or analyzed data, and sends information 114 to the indicator 111, the one or more personal devices 112, and/or the server 106.

From the sensor signals 113, the processor 123 of the control hub 104 can determine if methane gas is present around one of the one or more methane sensors 103 based on the measured amount of methane gas present around the one or more methane sensors 103 in volume measurements and/or ratio measurements of methane gas to air in the environment around the one or more methane sensors 103. The processor 123 may also determine which of the one or more methane sensors 103 sent the sensor signals 113.

The processor 123 can analyze the decoded data, including converting the raw data from one unit, such as ppm, to another unit, such as SCFH. The processor 123 may determine whether there is a methane leak near the one or more methane sensors 103. The processor 123 may also determine how large or serious the methane leak is based on methane volume and/or ratio of methane gas to air at discrete time increments or total methane gas over time, averaging a volume of methane measured over a time period. The processor 123 may also determine if the measurements made by the one or more methane sensors 103 are within a range indicative of a false reading or within an error range. Finally, the processor 123 may also determine important data characterizations such as maximum, minimum, mean, median, and/or mode of measured methane.

The processor 123 is configured to determine the location of the measured methane from the sensor signals 113. The processor 123 determines the location of the measured methane by comparing the identity of the one or more methane sensors 103 received in the sensor signals 113 and comparing the identity with a known location of each of the one or methane sensors 103. The processor 123 is configured to store information 114 on the memory 122 of the control hub 104 whether the information 114 is analyzed data or data from the sensor signals 113 in its raw form. The processor 123 is then configured to wirelessly communicate the information 114, whether the information 114 is analyzed data or data from the sensor signals 113 in its raw form, to the indicator 111, one or more personal devices 112, and/or the server 106.

The information 114 includes, but is not limited to, the presence of methane in the environment around the one or more methane sensors 103, the measured amount of methane in the environment around the one or more methane sensors 103 in volume and/or ratio, the location of the measured amount of methane, the presence of a methane leak, the location of the methane leak, or any combination of the data analyzed by the processor 123 of the control hub 104.

The processor 123 can also be configured to only communicate the information 114 when the measured and/or analyzed amount of methane has surpassed a minimum threshold. That minimum threshold may be stored in the control hub 104, the server 106, and/or may be input by a user or operator and may be as low as 0 ppm.

The indicator 111 is located at or near the worksite 101 and is viewable by workers at the worksite 101. The indicator 111 may be located out of hazardous areas and in close proximity to decision makers and workers at the worksite 101. The indicator 111 is in wireless communication with the control hub 104. The indicator 111 comprises a visual indication 126, such as lights or a strobe. The indicator 111 also comprises an audible indication 127, such as a siren or speakers. The indicator 111 receives the information 114 from the control hub 104. In response to receiving the information 114 from the control hub 104, the indicator 111 is configured to indicate, by audible indication 127 or visual indication 126, the presence of, amount of, and/or location of the measured amount of methane.

The indicator 111 also includes a repeater 125 and may be in wireless communication with the one or more methane sensors 103. The repeater 125 is used to assist in wireless communication between the one or more methane sensors 103 and the control hub 104 if the one or more methane sensors 103 are out of range of typical wireless communication with the control hub 104. Thus, the indicator 111 receives the sensor signals 113 from the one or more methane sensors 103. In response to receiving the sensor signals 113 from the one or more methane sensors 103, the indicator 111 is configured to indicate, by audible indication 127 or visual indication 126, the presence of, amount of, and/or location of the measured amount of methane received from the sensor signals 113, and repeat the sensor signals 113 to the control hub 104 to assist in the communication between the one or more methane sensors 103 and the control hub 104.

The indicator 111 may be configured to only indicate the presence of, amount of and/or location of the measured amount of methane if the one or more methane sensors 103 and/or the control hub 104 communicate that the measured and/or analyzed amount of methane has surpassed a minimum threshold. That minimum threshold may be stored in the indicator 111, the control hub 104, or may be input by a user or operator and may be as low as Oppm.

The one or more personal devices 112 may include, but are not limited to, a handheld device, a wearable device, a cellphone, a personal computer, and/or any combination thereof. The one or more personal devices 112 receive information 114 from the control hub 104 and/or sensor signals 113 from the one or more methane sensors 103 and are configured to indicate, by audible indication, visual indication, or indication on a user interface, the presence of, amount of, and/or location of the measured amount of methane to the user, wearer, and/or operator of the one or more personal devices 112.

The server 106 is a remote server. The server 106 may be a cloud server and is hosted at a location somewhere other than the worksite 101. The server 106 is configured to wirelessly communicate with the control hub 104. The server 106 is configured to receive the information 114 from the control hub 104, analyze the information 114 received from the control hub 104, store the data received from the control hub 104, and make accessible the information 114 and data stored in the server 106.

The analysis done by the server 106 includes, but is not limited to the capabilities of the control hub 104 referenced above. The analysis further includes generating visuals, graphics, charts, comparisons, reports, or any combination thereof. In generating these tools, the server 106 may compare the data, including the measured amount of methane gas, to standards such as safety standards, regulatory standards, and/or environmental standards.

The server 106 may also store the data to be accessed at another time. The data stored may include the sensor signals 113 data in its raw form, the analyzed data from the control hub 104, and/or data that has been further analyzed by the server 106.

The data stored on the server 106 may be manually accessed by users or the data may be automatically pushed to users. The data stored may also be accessed by another server or a computing device. The server 106 is also configured to send the stored information 114 back to the control hub 104, the indicator 111, or the one or more personal devices 112.

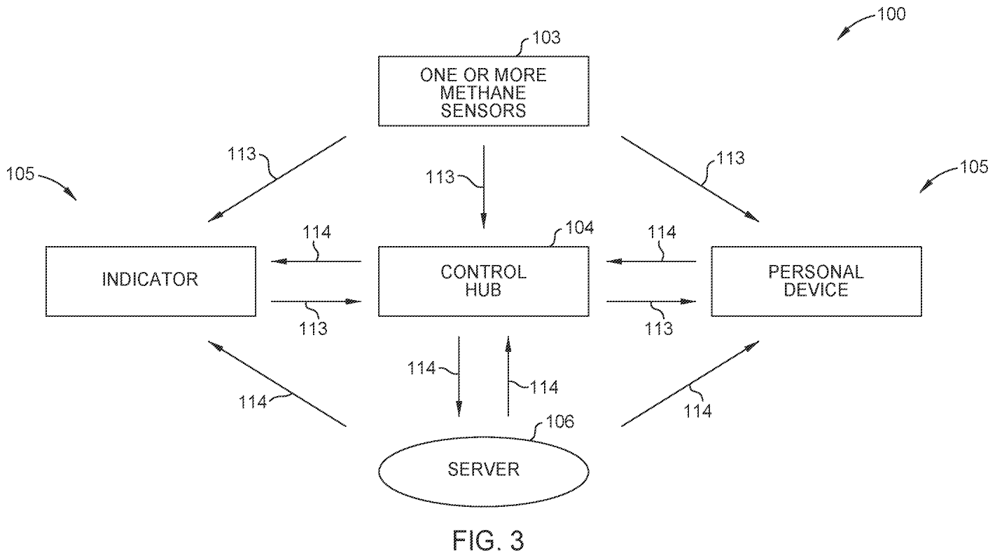

FIG. 3 illustrates the methane monitoring system 100, of FIG. 1 according to one embodiment.

The methane monitoring system 100 comprises the one or more methane sensors 103, the control hub 104, the indicator 111, the one or more personal devices 112, and the server 106. The one or more methane sensors 103 measure an amount of methane around equipment (such as equipment 102 of FIG. 1) at a worksite (such as worksite 101 of FIG. 1). The one or more methane sensors 103 then communicate measured data to the control hub 104, the indicator 111, and the one or more personal devices 112 by sensor signals 113.

The sensor signal 113 sent by the one or more methane sensors 103 includes, but is not limited to, a presence, or lack thereof, of methane in the environment around the one or more methane sensor 103, the measured amount of methane in the environment around the one or more methane sensor 103, an indication as to which of the one or more methane sensors 103 is sending the sensor signal 113, or any combination thereof.

In one or more embodiments, emission sampling is taken every 10 seconds from the methane sensors 103 then after one minute of sampling, the emission sampling data is averaged and sent to the server 106 and logged then distributed.

The sensor signal 113 is communicated by an antenna, (e.g. 4.3 dbi J-Pole with approximate omni-directional pattern), with a radio band of 2.4-2.5 Ghz ISM using a Zigbee Network Protocol. The wireless range may be 300 feet in an urban environment and 2 miles line of site range.

The control hub 104 receives the sensor signal 113, decodes the data from the sensor signal 113, analyzes the data from the sensor signal 113, stores the data from the sensor signal 113, and sends information 114 including the raw data from the sensors signal 113 and/or analyzed data to the indicator 111, the one or more personal devices 112 and the server 106.

The information 114 sent by the control hub 104 includes, but is not limited to the presence of methane in the environment around the one or more methane sensors 103, the measured amount of methane in the environment around the one or more methane sensors 103 in volume and/or ratio, the location of the measured amount of methane, the presence of a methane leak, the location of the methane leak, or any combination of the data analyzed by the processor (such as processor 123 of FIG. 2) of the control hub 104.

In one or more embodiments, the control hub 104 may not send this information 114 unless the measured and/or analyzed amount of methane received in the sensor signals 113 surpasses some minimum threshold.

The information 114 may be sent to the server 106, the indicator 111, and the one or more personal devices 112 by an antenna (e.g. 9 dBi Omni-directional) with a radio band of 2.4 GHz direct sequence spread spectrum ISM Band using a Zigbee Network Protocol and may have a range of up to 300 feet in urban environments and 2 miles line of site. The information 114 may also be sent via selectable satellite/WIFI connectivity. The information 114, may also be communicated by a wired connection.

The indicator 111, and the one or more personal devices 112 receive the sensor signal 113 from the one or more methane sensors 103 and repeat the sensor signal 113 to the control hub 104. When the sensor signal 113 is repeated to the control hub 104 from the indicator 111, it is repeated by with a radio band of 2.4 GHz direct sequence spread spectrum ISM Band using a Zigbee Network Protocol and may have a range of up to 300 feet in urban environments and 2 miles line of site.

The indicator 111 and the one or more personal devices 112 also receive the information 114 from the control hub 104 and indicate the information 114 received by the control hub 114. The server 106 receives the information 114 from the control hub 104. The server then can analyze the data included in the information 114, store the data included in the information 114, and make accessible the data included in the information 114.

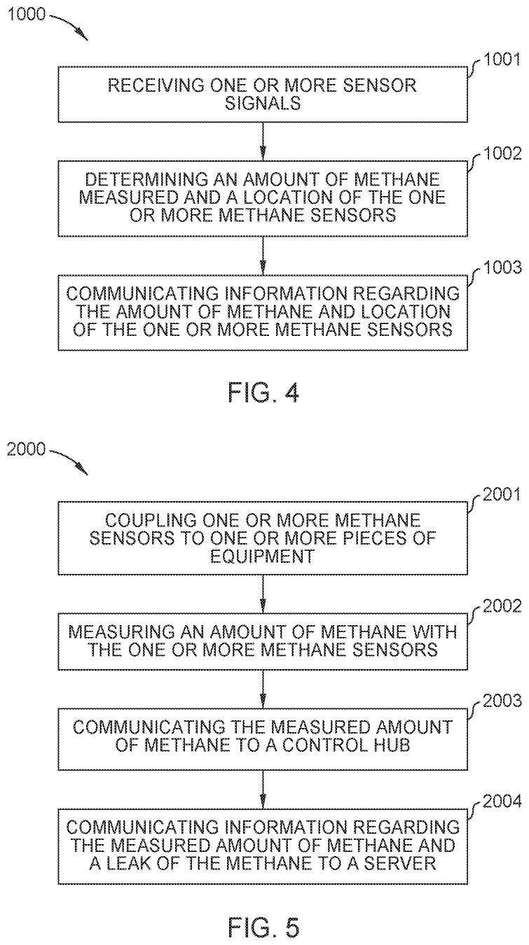

FIG. 4 illustrates a method 1000 for monitoring methane, according to one embodiment.

The method 1000 for monitoring methane comprises a step 1001 of a control hub (such as control hub 104 of FIG. 1) receiving one or more sensor signals (such as the one or more sensor signals 113 of FIG. 2) from one or more methane sensors (such as the one or more methane sensors 103 of FIG. 1). The one or more methane sensors comprise an internal nondispersive infrared sensor (such as NDIR sensor 117 of FIG. 2). The one or more methane sensors are coupled to one or more pieces of equipment (such as the one or more pieces of equipment 102 of FIG. 1). The signals correspond to methane measurements taken by the one or more methane sensors.

The control hub may receive the sensor signals wirelessly by an antenna (e.g. 9 dBi Omni-directional) with a radio band of 2.4 GHz direct sequence spread spectrum ISM Band using a Zigbee Network Protocol and may have a range of up to 300 feet in urban environments and 2 miles line of site.

The methane measurements taken by the one or more methane sensors may include detecting the presence of methane gas in the environment around the one or more methane sensors or may include volume measurements of methane in the environment surrounding the one or more methane sensors and/or ratio measurements of methane gas to air in the environment around the one or more methane sensors. The one or more methane sensors may be certified as Class 1, Div. 1.

The method 1000 further comprises a step 1002 of the control hub determining an amount of methane measured and a location of the one or more methane sensors measuring the amount of methane based on the one or more sensor signals from the one or more methane sensors. Determining the amount of methane may include analyzing the one or more sensor signals including, but not limited to, the capabilities of the processor of the control hub.

Determining the location may comprise determining which of the one or more methane sensors sent the one or more sensor signals. The identity of which of the one or more methane sensors that sent the one or more sensor signals is included in the one or more sensor signals. Once the identity of the one or more methane sensors is determined, the control hub can access its stored memory to determine where that one or more methane sensor is located within a worksite (such as worksite 101 of FIG. 1).

The method 1000 further comprises a step 1003 of the control hub communicating information (such as information 114 of FIG. 2) regarding the amount of methane and the location of the one or more methane sensors to a server (such as server 106 of FIG. 1).

Communicating the information is done wirelessly and may be accomplished by methods including, but not limited to Zigbee Network Protocol.

In one or more embodiments, the control hub only communicates the information when the measured and/or analyzed amount of methane surpasses a minimum threshold. That minimum threshold may be stored in the control hub, the server, or may be input by a user or operator.

FIG. 5 illustrates a method 2000 for monitoring methane at a worksite (such as worksite 101 of FIG. 1), according to one embodiment.

The method 2000 comprises a step 2001 of coupling one or more methane sensors (such as one or more methane sensors 103 of FIG. 1) to one or more pieces of equipment (such as one or more pieces of equipment 102 of FIG. 1). The one or more methane sensors comprise an internal non-dispersive infrared sensor (such as NDIR sensor 117 of FIG. 2) configured to measure methane.

The one or more methane sensors are coupled magnetically to and disposed on the one or more pieces of equipment. The one or more methane sensors may be certified as Class 1, Div. 1.

The method 2000 further comprises a step 2002 of measuring an amount of methane with the one or more methane sensors. The methane measurements taken may include detecting the presence of methane gas in the environment around the one or more methane sensors or may include volume measurements of methane in the environment surrounding the one or more methane sensors and/or ratio measurements of methane gas to air in the environment around the one or more methane sensors.

The method 2000 further comprises a step 2003 of communicating the measured amount of methane to a control hub (such as control hub 104 of FIG. 1). The one or more methane sensors may communicate the measured amount of methane to the control hub through sensor signals (such as sensor signals 113 of FIG. 2).

In one or more embodiments, the control hub may not be within communication range of the one or more methane sensors. In such embodiments, an indicator) such as indicator 111 of FIG. 2) may receive the sensor signals and repeat them to the control hub to aid in communication from the one or more methane sensors and the control hub.

The method 2000 may further comprise determining a location of the one or more methane sensors measuring the measured amount of methane. The control hub determines the location of the one or more methane sensors measuring the measured amount of methane by determining which of the one or more methane sensors sent the sensor signal including the measured amount of methane. The identity of which of the one or more sensors that sent the one or more sensor signals is included in the one or more sensor signals. Once the identity of the one or more methane sensors is determined, the control hub can access its stored memory to determine where that one or more methane sensor is located within a worksite.

The control hub may also determine the location of a methane gas leak by determining which of the one or more methane sensors sent the sensor signal including the measured amount of methane. The location of the one or more methane sensors in combination with the fact that the one or more methane sensors is sending a sensor signal indicating the presence of a measured amount of methane, the control hub can determine the location of a leak.

The method 2000 further comprises a step 2004 of communicating information (such as information 114 of FIG. 2) regarding the measured amount of methane and a leak of the methane to a server (such as server 106 of FIG. 1). The information includes, but is not limited to, the presence of methane in the environment around the one or more methane sensors, the measured amount of methane in the environment around the one or more methane sensors in volume and/or ratio, the location of the measured amount of methane, the presence of a methane leak, the location of the methane leak, and/or any combination of the data analyzed by the processor of the control hub.

The control hub also communicates information to the indicator and/or one or more personal devices (such as one or more personal devices 112 of FIG. 1). The indicator and/or the one or more personal devices then may, in response to receiving the information, audibly and/or visually indicate the information received by the control hub. In one or more embodiments, the control hub may only communicate the information to the server, the indicator, and/or the one or more personal devices when the measured and/or analyzed amount of methane has surpassed a certain minimum threshold. After receiving the information from the control hub, the server then analyzes the information.

The processors 119, 123 may be general-purpose programmable computing systems comprising one or more power sources, arrays, circuits, signal processing units, and/or memory devices. The processors 119, 123 are configured to save, transmit, read, and/or execute instructions to and/or from the memory devices to implement one or more of the embodiments described herein. The server 106, the personal device 112, the indicator 111, and/or other components of the methane monitoring system 100 may also include processors similar to processors 119, 113.

Any one or more components of the methane monitoring system 100 may be integrally formed together, directly coupled together, and/or indirectly coupled together and are not limited to the specific arrangement of components illustrated in FIGS. 1-3. Any one or more of the embodiments of the methane monitoring system 100 and methods 1000 and 2000 may be combined in whole or part with any one or more of the embodiments of the methane monitoring system 100 and methods 1000 and 2000.

It will be appreciated by those skilled in the art that the preceding embodiments are exemplary and not limiting. It is intended that all modifications, permutations, enhancements, equivalents, and improvements thereto that are apparent to those skilled in the art upon a reading of the specification and a study of the drawings are included within the scope of the disclosure. It is therefore intended that the following appended claims may include all such modifications, permutations, enhancements, equivalents, and improvements. The disclosure also contemplates that one or more aspects of the embodiments described herein may be substituted in for one or more of the other aspects described. The scope of the disclosure is determined by the claims that follow.

I claim:

1. A methane monitoring system, comprising:
one or more methane sensors coupled to one or more pieces of equipment, the one or more methane sensors comprising an internal non-dispersive infrared sensor configured to measure an amount of methane;
a control hub in communication with the one or more sensors, wherein the control hub is configured to receive a sensor signal corresponding to the measured amount of methane from the one or more methane sensors; and
a server in communication with the control hub, wherein the server is configured to receive information regarding the measured amount of methane from the control hub.

2. The methane monitoring system of claim 1, wherein the one or more methane sensors are magnetically coupled to the one or more pieces of equipment.

3. The methane monitoring system of claim 1, wherein the one or more methane sensors are in wireless communication with the control hub and the control hub is in wireless communication with the server.

4. The methane monitoring system of claim 1, wherein the internal non-dispersive infrared sensor is configured to measure an amount of methane.

5. The methane sensing system of claim 1, wherein the control hub is configured to determine a location of the one or more methane sensors measuring the amount of methane.

6. The methane monitoring system of claim 5, wherein:
the control hub is configured to communicate the location of the one or more methane sensors measuring the amount of methane; and
the server is configured to determine a location of a methane leak and a volume of the methane leak based on the location of the one or more methane sensors and the measured amount of methane.

7. The methane monitoring system of claim 1, further comprising an external device, wherein at least one of the one or more methane sensors, the control hub, and the server are configured to communicate with the one or more external devices.

8. The methane monitoring system of claim 5, wherein the external device is an indicator, wherein:
at least one of the one or more methane sensors, the control hub, and the server are configured to communicate the measured amount of methane with the indicator; and
the indicator is configured to visually or audibly signal the measured amount of methane.

9. The methane monitoring system of claim 1, wherein the control hub is configured to only communicate the measured amount of methane when the measured amount of methane is above a minimum threshold.

10. A method for monitoring methane, comprising:
receiving one or more sensor signals from one or more methane sensors comprising an internal nondispersive infrared sensor, the one or more methane sensors being coupled to one or more pieces of equipment, wherein the signals correspond to methane measurements taken by the one or more methane sensors;
determining an amount of methane measured and a location of the one or more methane sensors measuring the amount of methane based on the one or more sensor signals from the one or more methane sensors; and
communicating information regarding the amount of methane and the location of the one or more methane sensors to a server.

11. The method of claim 10, wherein communicating the measured amount of methane to the server occurs when the measured amount of methane is above a minimum threshold.

12. A method for monitoring methane at a worksite comprising:
coupling one or more methane sensors to one or more pieces of equipment, the one or more methane sensors comprising an internal non-dispersive infrared sensor configured to measure methane;
measuring an amount of methane with the one or more methane sensors;
communicating the measured amount of methane to a control hub; and
communicating information regarding the measured amount of methane and a methane leak to a server.

13. The method of claim 12, wherein the one or more methane sensors are magnetically coupled the one or more pieces of equipment.

14. The method of claim 12, wherein the internal non-dispersive infrared sensor is configured to measure an amount of methane.

15. The method of claim 12, further comprising determining a location of the one or more methane sensors measuring the amount of methane.

16. The method of claim 15, further comprising:
communicating the location of the one or more methane sensors measuring the amount of methane; and
determining a location of the methane leak and a volume of the methane leak using the location of the one or more methane sensors measuring the amount of methane and the measured amount of methane.

17. The method of claim 12, wherein communicating the measured amount of methane to the server occurs when the measured amount of methane is above a minimum threshold.

18. The method of claim 12, further comprising communicating the measured amount of methane to an external device, wherein the external device receives the measured amount of methane from one or more of the one or more methane sensors, the control hub, and the server.

19. The method of claim 18, further comprising:
communicating the measured amount of methane to the external device; and
visually or audibly indicating the measured amount of methane with the external device.

20. The method of claim 12, wherein the one or more methane sensors measure an amount of methane in parts per million, and the method further comprises converting the measured amount of methane from parts per million into standard cubic feet per hour.
